(19) United States
(12) Patent Application Publication
(10) Pub. No.: US 2026/0216676 A1
(43) Pub. Date: Jul. 30, 2026

(54) METHOD FOR PRILLING UREA AND UREA PRILLING APPARATUS

(71) Applicant: Toyo Engineering Corporation, Chiba-shi, Chiba (JP)

(72) Inventors: Keishi SANO, Chiba-shi, Chiba (JP); Masaki HIGAKI, Chiba-shi, Chiba (JP); Yuichiro WAKASHIMA, Chiba-shi, Chiba (JP)

(21) Appl. No.: 19/149,668

(22) PCT Filed: Mar. 28, 2024
(86) PCT No.: PCT/JP2024/012599

§ 371 Date: Jul. 21, 2025

(30) Foreign Application Priority Data
Mar. 30, 2023 (JP) 2023-055131

(51) Int. Cl. B01J 2/04; C07C 275/02

(52) U.S. Cl. CPC B01J 2/04; C07C 275/02

(57) ABSTRACT

A method for prilling urea using a urea prilling tower including an exhaust port provided at a tower top, a spraying portion for molten urea, atmosphere inlet ports and a collection portion is provided. The spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction. In a process of spraying the molten urea atmosphere is taken in at an ambient temperature through the atmosphere inlet ports to cool and solidify the molten urea to form the urea particles, and heating air is supplied to heat the interior of the urea prilling tower.

### FIELD OF THE INVENTION

The present invention relates to a method for prilling urea, and a urea prilling apparatus suitable for performing the prilling method.

As described in FIG. 1 and paragraphs [0006] to [0007] of JP-A 2016-500678, a prilling process using a prilling tower when producing urea prills is known. The claims of JP-A 2016-500678 describe inventions of a prilling tower and a prilling process in which the prilling tower of FIG. 1 is modified to enable improvements of overall efficiency and quality of the final product, and a prilling tower of FIGS. 2 and 3 is illustrated as a preferable embodiment.

Further, US-A 2002-0056931 and EP-A 3995487 each describe a method for taking in air to an interior of a prilling tower when producing urea prills in the prilling tower. Further, those provided with an acid scrubbing facility for processing exhaust air as an environmental measure are also known.

The problem to be solved by the present invention is to provide a method for prilling urea capable of reducing a moisture content of urea particles after production, and a urea prilling apparatus suitable for performing the prilling method.

The present invention provides a method for prilling urea using a urea prilling tower including an exhaust port provided at a tower top, a spraying portion for molten urea, a plurality of atmosphere inlet ports and a collection portion for produced urea particles, in which the urea prilling tower is configured such that the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction, wherein, in a process of spraying the molten urea in an interior of the urea prilling tower and taking in an atmosphere at an ambient temperature of the urea prilling tower through the atmosphere inlet ports upwardly from below to cool and solidify the molten urea to form the urea particles, heating air at a temperature higher than the ambient temperature is supplied in addition to the atmosphere at the ambient temperature to heat the interior of the urea prilling tower.

Further, the present invention provides a urea prilling apparatus including a urea prilling tower and a heating air supply device, in which the urea prilling tower includes an exhaust port provided at a tower top, a spraying portion for molten urea, a plurality of atmosphere inlet ports and a collection portion for prilled urea particles, and the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction, wherein the heating air supply device includes a heating air production portion and a heating air supply line for supplying heating air produced in the heating air production portion to an interior of the urea prilling tower.

The method for prilling urea of the present invention can produce urea particles with a low moisture content and stable quality.

### BRIEF DESCRIPTION OF THE DRAWINGS

FIG. 1 A front view of a urea prilling apparatus suitable for performing a method for prilling urea, provided that it is shown with an outer wall partially removed such that internal structures are visible.

FIG. 2 A cross-sectional view in a width direction of FIG. 1, provided that a heating air supply device is shown by a plan view.

FIG. 3 A partially enlarged view of FIG. 1.

FIG. 4 A partially enlarged view corresponding to FIG. 3 showing another embodiment of FIG. 1.

FIG. 5 A partially enlarged view corresponding to FIG. 3 showing another embodiment of FIG. 4.

FIG. 6 A partially enlarged view corresponding to FIG. 3 showing another embodiment of those shown in FIGS. 1 and 4.

FIG. 7 A partially enlarged view of a lengthwise cross section of a portion including an atmosphere inlet port shown in FIG. 6.

FIG. 8 A partially enlarged view corresponding to FIG. 3 showing another embodiment of those shown in FIGS. 1, 4 and 6.

FIG. 9 A partially enlarged view corresponding to FIG. 3 showing another embodiment of those shown in FIGS. 1, 4, 6 and 8.

### EMBODIMENTS OF THE INVENTION

<Method for Prilling Urea>

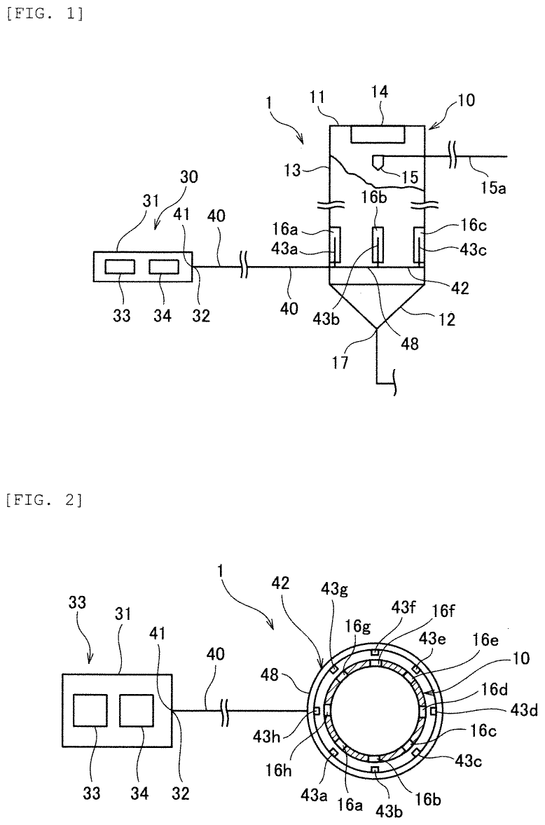

The method for prilling urea of the present invention can be performed using a publicly-known urea prilling tower shown in FIG. 1 or FIGS. 2 and 3 of JP-A 2016-500678 with facilities necessary to implement the present invention added to the publicly-known urea prilling tower. The publicly-known urea prilling tower includes an exhaust port provided at a tower top, a spraying portion for molten urea, a plurality of atmosphere inlet ports and a collection portion for prilled urea particles. An acid scrubbing facility for processing exhaust air is not required in the exhaust port provided at the tower top when the method for prilling urea of the present invention is performed, and thus, an acid scrubbing step for processing exhaust air is not required in the method for prilling urea, either. The spraying portion for molten urea is disposed on an upper side of the urea prilling tower, the plurality of atmosphere inlet ports are disposed on a lower side of the urea prilling tower, and the collection portion is disposed at a tower bottom on a further lower side of the atmosphere inlet ports. The plurality of atmosphere inlet ports are disposed at intervals in a circumferential direction. A planar shape of the plurality of atmosphere inlet ports is not particularly limited, but is preferably square or rectangular.

When a prilling method of a conventional art is performed using the publicly-known urea prilling tower, molten urea is sprayed in an interior of the urea prilling tower, and an atmosphere at an ambient temperature of the urea prilling tower is taken in through the atmosphere inlet ports upwardly from below to cool and solidify the molten urea to form urea particles. The same molten urea as a liquid phase (molten urea) supplied to a sprayer device in JP-A 2016-500678 can be used.

The present invention is characterized by including a step of supplying heating air at a temperature higher than the ambient temperature of the urea prilling tower in addition to the atmosphere at the ambient temperature to heat the interior of the urea prilling tower. Therefore, the implementation of the present invention requires a facility for producing the heating air and a facility for supplying the heating air to the urea prilling tower (which are the aforementioned facilities necessary to implement the present invention) in addition to the publicly-known urea prilling tower.

The atmosphere at the ambient temperature of the urea prilling tower is an atmosphere inside a plant in which the urea prilling tower is located (or an atmosphere outside the plant), and the atmospheric temperature varies according to the season or time period of a day (i.e., the temperature fluctuates). It is considered that, when the temperature decreases, for example, in winter or at night, the atmosphere taken in to the interior of the urea prilling tower through the atmosphere inlet ports and coming in contact with urea particles excessively cools the urea particles, and they absorb moisture in the atmosphere taken in during cooling, resulting in the occurrence of a problem of an increased moisture content of a product (urea particles after production). However, if the aforementioned step of supplying the heating air at a temperature higher than the ambient temperature to heat the interior of the urea prilling tower is provided, the occurrence of the aforementioned problem of an increased moisture content of the product (urea particles after production) can be prevented.

In a preferable embodiment of the method for prilling urea of the present invention, a temperature when an environmental temperature of surroundings in which the urea prilling tower is installed (the ambient temperature) is highest is considered to be an assumed highest design temperature of the urea prilling tower, and when the ambient temperature at which the urea prilling tower is installed is lower than the assumed highest temperature, the step of supplying the heating air at a temperature higher than the ambient temperature in addition to the atmosphere at the ambient temperature to heat the interior of the urea prilling tower can be performed.

The assumed highest design temperature of the urea prilling tower is a temperature when an environmental temperature of surroundings in which the urea prilling tower is installed (the ambient temperature) is highest, and is specifically a temperature during the summer daytime. Even during the summer daytime, the assumed highest design temperature (T1) of the urea prilling tower varies according to multiple factors such as an area, a surrounding environment of a location of the plant in which the urea prilling tower is installed, and others. When the ambient temperature (T2) at which the urea prilling tower is installed is lower than the assumed highest temperature (T1) (T2<T1), the heating air at a temperature (T3) higher than the ambient temperature (T2) is supplied in addition to the atmosphere at the ambient temperature (T2) to heat the interior of the urea prilling tower. At that time, the relation between T1 and T3 is T3≥T1.

When the step of supplying the heating air at the temperature (T3) higher than the ambient temperature (T2) in addition to the atmosphere at the ambient temperature (T2) is performed, any one step selected from the following steps is preferably performed,
(a) a step of supplying the heating air from the outside of the atmosphere inlet ports while taking in the atmosphere through the atmosphere inlet ports to the interior,
(b) a step of supplying part of the heating air from the outside of the atmosphere inlet ports and supplying the rest of the heating air directly to the inside of the atmosphere inlet ports while taking in the atmosphere through the atmosphere inlet ports to the interior, and
(c) a step of supplying the heating air directly to the inside of the atmosphere inlet ports while taking in the atmosphere through the atmosphere inlet ports to the interior.

When the step of (a) is performed, it takes the form of supplying the heating air to the interior of the urea prilling tower while mixing it with the atmosphere taken in through the atmosphere inlet ports.

When the step of (b) is performed, it takes the form of (a) for part of the heating air, and it also includes, for the rest of the heating air supplied directly to the interior of the urea prilling tower, the form of mixing the rest of the heating air with the atmosphere taken in in the interior of the urea prilling tower.

When the step of (c) is performed, it takes the form of mixing all the heating air with the atmosphere taken in in the interior of the urea prilling tower.

When the step of (a) or (b) is performed, one step selected from the following steps is preferably performed,
(ab-1) a step of supplying the heating air from a plurality of positions opposed to the plurality of atmosphere inlet ports when supplying the heating air from the outside of the atmosphere inlet ports, and
(ab-2) a step of supplying the heating air from a plurality of positions outside the urea prilling tower and between the plurality of atmosphere inlet ports towards adjacent atmosphere inlet ports on both sides when supplying the heating air to the atmosphere inlet ports.

When the step of (ab-1) is performed, for example, a heating air supply line (main line) and a plurality of branch lines branched therefrom can be used such that heating air supply ports of the branch lines are disposed opposite to the atmosphere inlet ports to perform the step of supplying the heating air from the plurality of positions opposed to the atmosphere inlet ports. When the step of (ab-2) is performed, for example, a heating air supply line (main line) and a plurality of branch lines branched therefrom can be used such that heating air supply ports of the plurality of branch lines are positioned between the adjacent atmosphere inlet ports, and their shape, size and formation position are adjusted to allow the heating air supply ports to release the heating air in the directions of the adjacent atmosphere inlet ports to perform the step of supplying the heating air.

A technique for supplying the heating air is not particularly limited, and for example, a heating air production device provided with a heater or a heat exchanger for heating the atmosphere at the ambient temperature (T2) and also combined as necessary with a blowing device for supplying the heating air can be used to supply the heating air. At that time, the heating air production device is connected to the urea prilling tower via a duct or the like such that the heating air can be supplied to the urea prilling tower through the duct.

The aforementioned embodiments for supplying the heating air are more preferably performed considering an operation load factor in the urea prilling tower (a percentage of an average urea particle production amount during a period to a maximum urea particle production amount during the same period). For example, when the same amount of the heating air at the same temperature is supplied to the interior of the urea prilling tower (for example, supplied by any of the aforementioned embodiments (a) to (c)) at operation load factors of 100% and 50%, while a heating condition in the interior of the urea prilling tower is sufficient in operation conditions at an operation load factor of 100%, the heating condition in the interior of the urea prilling tower becomes insufficient in operation conditions at an operation load factor of 50%, leading to more reduction in the temperature of the interior of the urea prilling tower, which may result in the occurrence of the problem of an increased moisture content of the product (urea particles after production). Therefore, when the operation load factor in the urea prilling tower is a low value (Lₓ) (%) as compared to a maximum value (Lₘₐₓ) (%), a step of increasing a supply amount of the heating air as a difference of Lₘₐₓ−Lₓ (%) increases is preferably performed. For example, when a supply amount of the heating air (temperature T° C.) to the interior of the urea prilling tower when Lₘₐₓ=100% is V, a supply amount of the heating air (temperature T° C.) when Lₓ=50% can fall within the range of 1V to 5V. Such implementation suppresses a temperature reduction in the interior of the urea prilling tower, such that an increase in the moisture content of the product (urea particles after production) can be prevented, and the moisture content can be kept low.

While the product (urea particles after production) satisfying any of the following first, second and third requirements can be produced by performing the method for prilling urea of the present invention, it is preferable to be able to produce the product (urea particles after production) satisfying the third requirement,
first requirement: a temperature of the urea particles immediately after collection from the collection portion of the urea prilling tower is 40° C. or more,
second requirement: a moisture content of the urea particles immediately after collection from the collection portion of the urea prilling tower (measurement method: measuring with a Karl Fischer moisture meter or heating an analysis sample with a drier and measuring by loss on drying) is 0.3% or less, and
third requirement: both the first and second requirements are satisfied.

### <Urea Prilling Apparatus>

The urea prilling apparatus of the present invention is explained with reference to FIGS. 1 to 9. The urea prilling apparatus of the present invention is an apparatus suitable for performing the aforementioned method for prilling urea. As shown in FIG. 1, a urea prilling apparatus 1 is provided with a urea prilling tower 10 and a heating air supply device 30.

The urea prilling tower 10 includes an outer wall (casing) provided with a tower top 11, a tower bottom 12 and a tower side wall 13, and an upper portion of the casing is shown in FIG. 1 with the tower side wall 13 removed such that the internal structures are visible. The tower bottom 12 can have a conical shape as shown in the figure to facilitate collection of prilled urea particles. While a cross-sectional shape in a width direction (direction orthogonal to a height direction) of the urea prilling tower 10 is preferably circular, it is not limited thereto and can be polygonal such as quadrangular, hexagonal or the like and others.

An exhaust port 14 is provided at the tower top 11. A spraying portion 15 for molten urea is provided on an upper side of the urea prilling tower 10 and below the tower top 11, and connected to a supply portion (not shown) for molten urea via a supply line 15a for molten urea. A plurality of atmosphere inlet ports 16 (16a to 16h) are provided on a lower side of the urea prilling tower 10. The plurality of atmosphere inlet ports 16 are disposed at intervals in a circumferential direction. A collection portion 17 for urea particles prilled in an interior of the urea prilling tower 10 is disposed at the tower bottom 11 on a further lower side of the atmosphere inlet ports 16 of the urea prilling tower 10.

The size of the urea prilling tower 10 can be appropriately determined considering production ability, location conditions or the like, and for example, the urea prilling tower 10 can have a columnar outer shape with a height of 50 m to 150 m and a diameter of 10 m to 30 m, and the atmosphere inlet ports 16 can each have a size of 3 m×9 m.

The heating air supply device 30 includes a heating air production portion 31 and a heating air supply line 40 for supplying heating air produced in the heating air production portion 31 to the interior of the urea prilling tower 10. The heating air production portion 31 provided with a heating device 33 for an atmosphere taken in and a blowing device 34 for blowing the heated atmosphere can be used. As the heating device 33, for example, a heater or a heat exchanger can be used. The heating air supply line 40 includes a duct capable of supplying the heating air produced in the heating air production portion 31 to the urea prilling tower 10. The heating air supply line 40 is configured such that a heating air discharge port 32 of the heating air production portion 31 is connected to a first end opening 41 of the heating air supply line 40, and a second end opening 48 of the heating air supply line 40 is connected to the plurality of atmosphere inlet ports 16 of the urea prilling tower 10.

In an embodiment shown in FIGS. 1 to 3, the second end opening 48 of the heating air supply line 40 includes an annular portion 42 and a plurality of heating air discharge portions 43 (43a to 43h) provided vertically from the annular portion 42. The heating air discharge portions 43 (43a to 43h) each include a hollow columnar portion 44 and a heating air discharge port 45 formed on a surface spaced and opposed to each of the atmosphere inlet ports 16 (16a to 16h) of the urea prilling tower 10. A cross-sectional shape in a width direction of the hollow columnar portions 44 can be circular, polygonal, oval or the like, but is not limited thereto.

The heating air discharge port 45 may be a slit-like opening or a group of pores. In the embodiment shown in FIG. 1 to 3, the heating air discharge port 45 is spaced and opposed to each of the atmosphere inlet ports 16 (16a to 16h), such that the heating air is supplied to an interior of the atmosphere inlet ports 16 (16a to 16h) from the outside of the urea prilling tower 10.

The embodiment of FIGS. 1 to 3 can also be an embodiment in which part of or all the heating air discharge portions 43 (43a to 43h) are disposed to be incorporated into the interior of the atmosphere inlet ports 16 (16a to 16h) such that the heating air is discharged directly to the interior of the urea prilling tower 10. When part of the heating air discharge portions 43 (43a to 43h) are disposed to be incorporated into the interior of the atmosphere inlet ports 16 (16a to 16h), part of the heating air is supplied to the atmosphere inlet ports 16 (16a to 16h) from the outside of the urea prilling tower 10, and the rest thereof is discharged directly to the interior of the urea prilling tower 10. When all the heating air discharge portions 43 (43a to 43h) are disposed to be incorporated into the interior of the atmosphere inlet ports 16 (16a to 16h), all the heating air is discharged directly to the interior of the urea prilling tower 10.

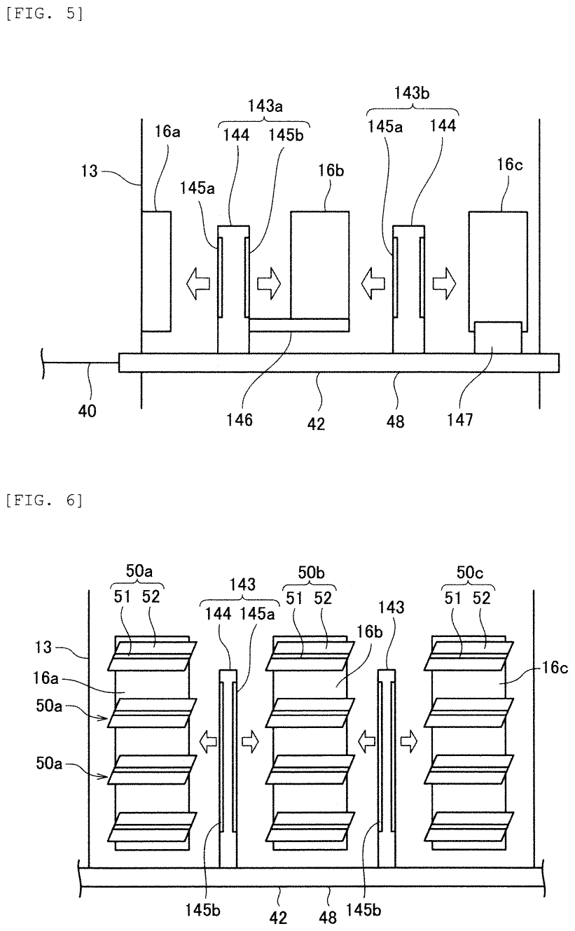

Next, an embodiment of FIG. 4 is explained together with FIGS. 1 and 2. Note that all the following embodiment of FIG. 4 and embodiments of FIGS. 5, 6, 8 and 9 show only part of the atmosphere inlet ports 16 (16a to 16h) in the figures, but include the atmosphere inlet ports in the same manner as in the embodiment of FIGS. 1 to 3. The embodiment of FIG. 4 is the same as the embodiment of FIGS. 1 to 3 except that heating air discharge portions 143 (143a to 143b) in the embodiment of FIG. 4 are disposed in a different form from those of the heating air discharge portions 43 (43a to 43c) of the embodiment in FIGS. 1 to 3. The heating air discharge portions 143 (143a to 143b) are each provided vertically from the annular portion 42 such that it is located at a circumferentially midpoint position between adjacent atmosphere inlet ports 16 (16a to 16c). While the heating air discharge portions 143 (143a to 143b) may be spaced and opposed or contacted and opposed to the tower side wall 13, the heating air discharge portions 43 are spaced and opposed thereto in the embodiment shown in FIGS. 1 to 3. The heating air discharge portions 143 (143a to 143b) each include a hollow columnar portion 144, and slit-like or porous heating air discharge ports 145 at two positions opposed to one another in a horizontal direction of the hollow columnar portion 144 (direction along the tower side wall 13). A cross-sectional shape in a width direction of the hollow columnar portion 144 can be circular, polygonal, oval or the like, but is not limited thereto. In the embodiment shown in FIG. 4, two heating air discharge portions 143 can also be disposed between adjacent atmosphere inlet ports (16a and 16b or 16b and 16c). At that time, the slit-like or porous heating air discharge port 145 can be formed at only one position near each of the atmosphere inlet ports. In the embodiment of FIG. 4, the heating air discharged from the heating air discharge ports 145 (145a to 145b) is supplied to an interior of the atmosphere inlet ports 16 (16a to 16c) from the outside of the urea prilling tower 10.

Further, when the embodiment shown in FIG. 4 is considered to be a first form, the urea prilling apparatus can include a second or third form as shown in FIG. 5 in addition to the first form. Note that, while FIG. 5 shows both the second and third forms, an embodiment of only the second form or an embodiment of only the third form may be implemented. The second form shown in FIG. 5 includes a second hollow columnar portion 146 including an opening (not shown) which is branched from the heating air discharge portion 143a and extended in a direction along the annular portion 42. At that time, the second hollow columnar portion 146 may be extended from one heating air discharge portion 143 in any one direction or both directions of the atmosphere inlet ports 16. For example, it may be extended from the heating air discharge portion 143a in any one direction or both directions of the atmosphere inlet ports 16a and 16b. Further, it may be extended from the heating air discharge portion 143b in any one direction or both directions of the atmosphere inlet ports 16b and 16c. The second hollow columnar portion 146 has the opening at a position adjusted to allow the heating air to be discharged from a lower side (a side near the annular portion 42) of the atmosphere inlet ports 16a to 16c directly to the interior of the atmosphere inlet ports 16a to 16c. When the heating air is supplied to the interior of the atmosphere inlet ports 16a to 16c, the heating air (warm light air) is more likely to be taken in from an upper side (a side far from the annular portion 42) of the atmosphere inlet ports 16a to 16c to the interior of the urea prilling tower 10. When the second form of FIG. 5 is implemented, the heating air is taken in through the entire atmosphere inlet ports 16a to 16c.

The third form shown in FIG. 5 is a form in which a plurality of third hollow columnar portions 147 including an opening (not shown) are provided vertically from the annular portion 42 with the openings of the third hollow columnar portions 147 disposed opposite to the atmosphere inlet ports 16a to 16c. The third hollow columnar portions 147 are each provided vertically from the annular portion 42 corresponding to a circumferentially midpoint position between the heating air discharge portions 143a to 143c, and adjusted such that the openings are disposed opposite to the atmosphere inlet ports 16a to 16c. The position of the third hollow columnar portion 147 is adjusted to allow the heating air to be discharged from the lower side (the side near the annular portion 42) of the atmosphere inlet ports 16a to 16c to the interior of the atmosphere inlet ports 16a to 16c. The implementation of the third form of FIG. 5 attains the same effect as that of the above second embodiment.

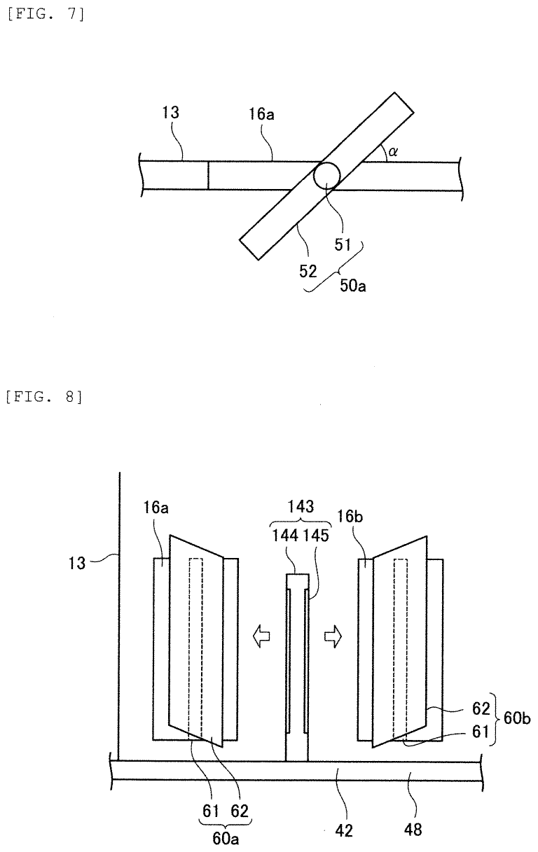

Next, an embodiment of FIG. 6 is explained together with FIGS. 1 to 4. Note that, also in the embodiment of FIG. 6, the second hollow columnar portion 146 or the third hollow columnar portion 147 can be provided in the same manner as in FIG. 5. The embodiment of FIG. 6 is different from the embodiment of FIG. 4 in that each of the atmosphere inlet ports 16 (16a to 16c) is provided with a plurality of adjustment tools 50a, 50b or 50c for adjusting an atmospheric inflow rate and inflow direction. A single adjustment tool 50a includes a rotation shaft 51 rotatably attached to both edges in a width direction of the atmosphere inlet port 16a, and a vane 52 fixed to the rotation shaft 51. A total of four adjustment tools 50a are mounted to the atmosphere inlet port 16a, and the four adjustment tools 50a all together serve as a louver.

The four attached adjustment tools 50a (louver) can all together increase or reduce an opening area of the atmosphere inlet port 16a in such a manner that the rotation of the rotation shaft 51 of each adjustment tool 50a changes an angle α (see FIG. 7) formed between a plane of the atmosphere inlet port 16a and a surface of the vane 52 between 0 degrees and 90 degrees. As a result, the inflow rate through the atmosphere inlet port 16a, the inflow direction through the atmosphere inlet port 16a to the interior or both the inflow rate and the inflow direction can be adjusted. Four adjustment tools 50b (louver) and four adjustment tools 50c (louver) are attached respectively to the atmosphere inlet ports 16b and 16c, and serve in the same manner as the adjustment tools 50a. A predetermined number of the same adjustment tools as the adjustment tools 50a are also attached to the rest of the atmosphere inlet ports not shown in the figure. In the embodiment shown in FIG. 6, two heating air discharge portions 143 can also be disposed between adjacent atmosphere inlet ports (16a and 16b or 16b and 16c). At that time, the slit-like or porous heating air discharge port 145 can be formed at only one position near each of the atmosphere inlet ports. In the embodiment of FIG. 6, the heating air discharged from the heating air discharge ports 145 is supplied to the interior of the atmosphere inlet ports 16 (16a to 16c) from the outside of the urea prilling tower 10.

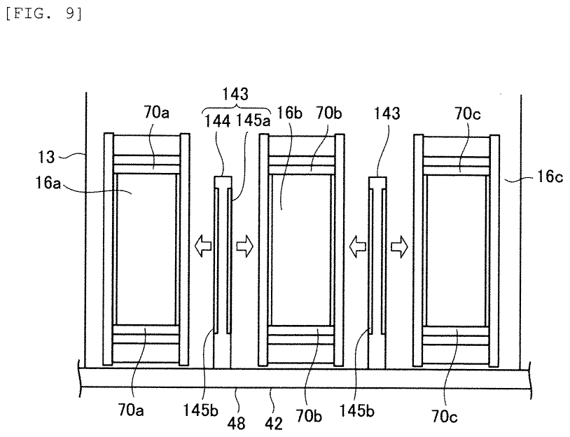

Next, an embodiment of FIG. 8 is explained together with FIGS. 1 to 6. Note that, also in the embodiment of FIG. 8, the second hollow columnar portion 146 or the third hollow columnar portion 147 can be provided in the same manner as in FIG. 5. The embodiment of FIG. 8 is different from the embodiment of FIG. 4 in that each of the atmosphere inlet ports 16 (16a to 16c) is provided with an adjustment tool 60a or 60b for adjusting an atmospheric inflow rate and inflow direction. The adjustment tool 60a includes a rotation shaft 61 rotatably attached to both ends in a lengthwise direction of the atmosphere inlet port 16a, and a vane 62 fixed to the rotation shaft 61. The adjustment tools 60a and 60b can all together increase or reduce an opening area of the atmosphere inlet ports 16a and 16b in such a manner that the rotation of each rotation shaft 61 changes an angle α (see FIG. 7) formed between a plane of the atmosphere inlet port 16a or 16b and a surface of the vane 62 between 0 degrees and 90 degrees. As a result, the inflow rates through the atmosphere inlet ports 16a and 16b, the inflow directions through the atmosphere inlet ports 16a and 16b to the interior or both the inflow rates and the inflow directions can be adjusted. In the embodiment shown in FIG. 8, two heating air discharge portions 143 can also be disposed between the adjacent atmosphere inlet ports (16a and 16b). At that time, the slit-like or porous heating air discharge port 145 can be formed at only one position near each of the atmosphere inlet ports. In the embodiment of FIG. 8, the heating air discharged from the heating air discharge ports 145 is discharged to the interior of the atmosphere inlet ports 16 (16a to 16b) from the outside of the urea prilling tower 10.

One or two or more shutters attached to each of the atmosphere inlet ports 16a to 16c can be used as adjustment tools 70a, 70b or 70c replacing the adjustment tools 50a, 50b or 50c or the adjustment tool 60a or 60b used in the embodiment shown in FIG. 6 or 8. As shown in FIG. 9, the one or two or more shutters are attached to, for example, one position on an upper edge side or a lower edge side of each of the atmosphere inlet ports 16a to 16c, or two positions on the upper edge side and the lower edge side, such that an opening area of the atmosphere inlet ports 16a to 16c can be increased or reduced by raising or lowering the shutters.

According to multiple factors such as a place of installation, a processing amount and others, in an embodiment, the urea prilling apparatus 1 can include any one selected from:
(A) a combination of a single urea prilling tower 10 and a single heating air supply device 30;
(B) a combination of a single urea prilling tower 10 and a plurality of heating air supply devices 30;
(C) a combination of a plurality of urea prilling towers 10 and a single heating air supply device 30; and
(D) a combination of a plurality of urea prilling towers 10 and a plurality of heating air supply devices 30.

The urea prilling apparatus 1 preferably can implement an embodiment in which a temperature when an environmental temperature of surroundings in which the urea prilling tower 10 is installed (an ambient temperature) is highest is considered to be an assumed highest design temperature of the urea prilling tower 10, and when the ambient temperature at which the urea prilling tower 10 is installed is lower than the assumed highest temperature, in addition to an atmosphere at the ambient temperature, heating air at a temperature higher than the ambient temperature can be supplied from the heating air supply device 30 to heat the interior of the urea prilling tower 10.

When implementing the above embodiment, the urea prilling apparatus 1 preferably can further implement an embodiment in which, when an operation load factor in the urea prilling tower 10 (a percentage of an average urea particle production amount during a period to a maximum urea particle production amount during the same period) is a low value (Lₓ) (%) as compared to a maximum value (Lₘₐₓ) (%), a supply amount of the heating air from the heating air supply device 30 can be increased as a difference of Lₘₐₓ−Lₓ (%) increases.

When the urea prilling apparatus 1 is used for producing urea particles, it is preferable to be able to satisfy a first requirement that a temperature of the urea particles immediately after collection from the collection portion 17 of the urea prilling tower 10 can be 40° C. or more, a second requirement that a moisture content of the urea particles immediately after collection from the collection portion 17 of the urea prilling tower 10 (measurement method: measuring with a Karl Fischer moisture meter or heating an analysis sample with a drier and measuring by loss on drying) can be 0.3% or less, or a third requirement that both the first and second requirements are satisfied.

### <Urea Prilling Method Using Urea Prilling Apparatus 1 (Method For Operating Urea Prilling Apparatus 1)>

Next, a urea prilling method using the urea prilling apparatus 1 (i.e., a method for operating the urea prilling apparatus 1) is explained with reference to FIGS. 1 to 9. Molten urea is sprayed from the spraying portion 15 for molten urea to an internal space of the urea prilling tower 10. The same molten urea as the liquid phase (molten urea) supplied to the sprayer device in JP-A 2016-500678 can be used. When the molten urea is sprayed, an atmosphere at an ambient temperature of the urea prilling tower 10 is taken in through the atmosphere inlet ports 16a to 16h upwardly from below to cool and solidify the molten urea to form urea particles.

The atmosphere at the ambient temperature of the urea prilling tower 10 is an atmosphere inside of a plant in which the urea prilling tower 10 is located (or an atmosphere outside the plant), and the atmospheric temperature varies according to the season or time period of a day (i.e., the temperature fluctuates). It is considered that, when the temperature decreases, for example, in winter or at night, the atmosphere taken in to the interior of the urea prilling tower 10 through the atmosphere inlet ports and coming in contact with urea particles excessively cools the urea particles, and they absorb moisture in the atmosphere taken in during cooling, resulting in the occurrence of a problem of an increased moisture content of the product (urea particles after production). When the urea prilling method using the urea prilling apparatus 1 of FIGS. 1 to 9 is performed, in addition to the atmosphere at the ambient temperature, heating air at a temperature higher than the ambient temperature of the urea prilling tower 10 is supplied from the heating air supply device 30 to heat the interior of the urea prilling tower 10. If such a step of supplying the heating air at a temperature higher than the ambient temperature to heat the interior of the urea prilling tower 10 is provided, the occurrence of the aforementioned problem of an increased moisture content of the product (urea particles after production) can be prevented, and the moisture content can be kept low. The produced urea particles are recovered from the collection portion 17 to be a product of stable quality.

When the urea prilling method using the urea prilling apparatus 1 of FIGS. 1 to 9 is performed, a temperature when an environmental temperature of surroundings in which the urea prilling tower 10 is installed (the ambient temperature) is highest is considered to be an assumed highest design temperature of the urea prilling tower 10, and when the ambient temperature at which the urea prilling tower 10 is installed is lower than the assumed highest temperature, the step of supplying the heating air at a temperature higher than the ambient temperature in addition to the atmosphere at the ambient temperature to heat the interior of the urea prilling tower 10 can be performed.

The assumed highest design temperature of the urea prilling tower 10 is a temperature when an environmental temperature of surroundings in which the urea prilling tower 10 is installed (the ambient temperature) is highest, and is specifically a temperature during the summer daytime.

Even during the summer daytime, the assumed highest design temperature (T1) of the urea prilling tower 10 varies according to multiple factors such as an area, a surrounding environment of a location of the plant in which the urea prilling tower 10 is installed, and others. When the ambient temperature (T2) at which the urea prilling tower 10 is installed is lower than the assumed highest temperature (T1) (T2<T1), the heating air at a temperature (T3) higher than the ambient temperature (T2) is supplied in addition to the atmosphere at the ambient temperature (T2) to heat the interior of the urea prilling tower 10. At that time, the relation between T1 and T3 is T3≥T1.

The urea prilling method using the urea prilling apparatus 1 of FIGS. 1 to 9 is more preferably performed considering an operation load factor in the urea prilling tower 10 (a percentage of an average urea particle production amount during a period to a maximum urea particle production amount during the same period). When the operation load factor in the urea prilling tower 10 (a percentage of an average urea particle production amount during a period to a maximum urea particle production amount during the same period) is a low value (Lₓ) (%) as compared to a maximum value (Lₘₐₓ) (%), a supply amount of the heating air is increased as a difference of Lₘₐₓ−Lₓ (%) increases. For example, when the same amount of the heating air at the same temperature is supplied to the interior of the urea prilling tower 10 at operation load factors of 100% and 50%, while a heating condition in the interior of the urea prilling tower 10 is sufficient in operation conditions at an operation load factor of 100%, the heating condition in the interior of the urea prilling tower 10 becomes insufficient in operation conditions at an operation load factor of 50%, leading to more reduction in the temperature of the interior of the urea prilling tower 10, which may result in the occurrence of the problem of an increased moisture content of the product (urea particles after production). Therefore, when the operation load factor in the urea prilling tower 10 is a low value (Lₓ) (%) as compared to a maximum value (Lₘₐₓ) (%), the step of increasing a supply amount of the heating air as a difference of Lₘₐₓ−Lₓ (%) increases is preferably performed. For example, when a supply amount of the heating air (temperature T° C.) to the interior of the urea prilling tower when Lₘₐₓ=100% is V, a supply amount of the heating air (temperature T° C.) when Lₓ=50% can fall within the range of 1V to 5V. Such implementation suppresses a temperature reduction in the interior of the urea prilling tower 10, such that an increase in the moisture content of the product (urea particles after production) can be prevented, and the product with a suppressed moisture content can be obtained.

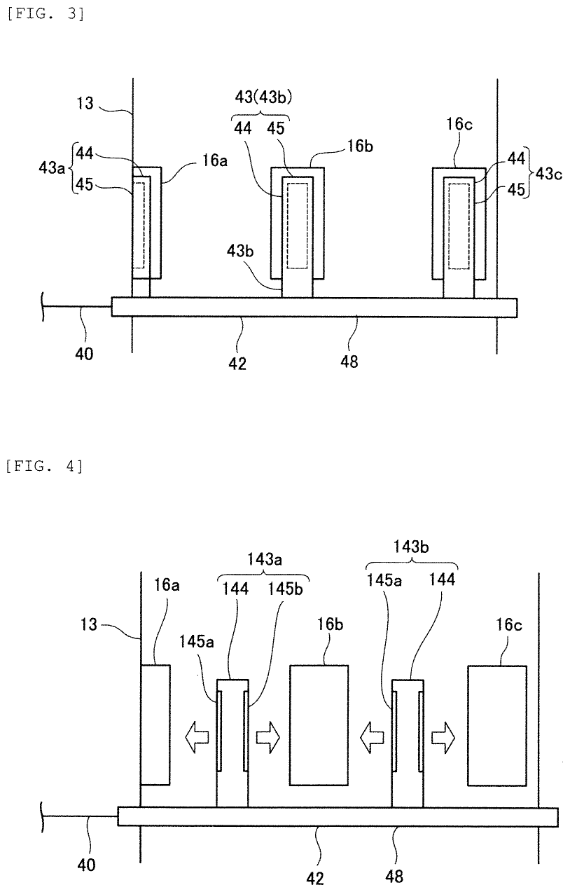

When each apparatus shown in FIG. 3, 4, 6 or 8 is used in the urea prilling method using the urea prilling apparatus 1, each apparatus supplies the heating air to the interior of the urea prilling tower 10 in a different embodiment from one another. In the apparatus shown in FIG. 3, the heating air supply line 40, the annular portion 42 and the heating air discharge portions 43 (43a to 43h) are used to supply the heating air produced in the heating air production portion 31 to the atmosphere inlet ports 16a to 16h, through which the heating air is supplied from the outside of the urea prilling tower 10 to the inside of the urea prilling tower 10. At that time, the heating air is supplied to the interior of the urea prilling tower 10 while outside air near the atmosphere inlet ports 16a to 16 h is also taken in.

In the apparatus shown in FIG. 4, the heating air supply line 40, the annular portion 42 and the heating air discharge portions 143 (143a to 143b) are used to supply the heating air produced in the heating air production portion 31 to the atmosphere inlet ports 16a to 16c, through which the heating air is supplied from the outside of the urea prilling tower 10 to the inside of the urea prilling tower 10. At that time, the heating air discharged from the slit-like or porous heating air discharge ports 145 of the heating air discharge portions 143 (143a to 143b) circumferentially moves along the tower side wall 13 while taking in surrounding outside air, and is supplied to the interior of the urea prilling tower 10 through the atmosphere inlet ports 16 (16a to 16c). The mixed air of the heating air and the outside air supplied to the interior of the urea prilling tower 10 is mixed with air in the interior of the urea prilling tower 10 and increases an internal temperature of the urea prilling tower 10.

In the apparatus shown in FIG. 6, the heating air supply line 40, the annular portion 42 and the heating air discharge portions 143 (143a to 143b) are used to supply the heating air produced in the heating air production portion 31 to the atmosphere inlet ports 16a to 16c, through which the heating air is supplied from the outside to the inside of the urea prilling tower 10. The heating air discharged from the slit-like heating air discharge ports 145 of the heating air discharge portions 143 (143a to 143b) circumferentially moves along the tower side wall 13 while taking in surrounding outside air, and is supplied to the interior of the urea prilling tower 10 through the atmosphere inlet ports 16 (16a to 16c). At that time, the inflow rate of the mixed air can be adjusted by operating the adjustment tools 50a to 50c attached to the atmosphere inlet ports 16a to 16c to increase or reduce the opening area of the atmosphere inlet ports 16a to 16c. The mixed air of the heating air and the outside air supplied to the interior of the urea prilling tower 10 is mixed with air in the interior of the urea prilling tower 10 and increases an internal temperature of the urea prilling tower 10.

In the apparatus shown in FIG. 8, the heating air supply line 40, the annular portion 42 and the heating air discharge portions 143 are used to supply the heating air produced in the heating air production portion 31 to the atmosphere inlet ports 16a to 16b, through which the heating air is supplied from the outside to the inside of the urea prilling tower 10. The heating air discharged from the slit-like or porous heating air discharge ports 145 of the heating air discharge portions 143 circumferentially moves along the tower side wall 13, and is supplied to the interior of the urea prilling tower 10 together with surrounding outside air through the atmosphere inlet ports 16 (16a to 16b).

Any requirement of the following first, second and third requirements can be satisfied by performing the urea prilling method using the urea prilling apparatus 1 of FIGS. 1 to 9,

### (First Requirement)

a temperature of the urea particles immediately after collection from the collection portion 17 of the urea prilling tower 10 can be 40° C. or more,

### (Second Requirement)

a moisture content of the urea particles immediately after collection from the collection portion 17 of the urea prilling tower 10 (measurement method: measuring with a Karl Fischer moisture meter or heating an analysis sample with a drier and measuring by loss on drying) can be 0.3% or less, or

### (Third Requirement)

both the first and second requirements are satisfied.

The present invention encompasses the inventions below.

(1) A method for prilling urea using a urea prilling tower including an exhaust port provided at a tower top, a spraying portion for molten urea, a plurality of atmosphere inlet ports and a collection portion for prilled urea particles, in which
the urea prilling tower is configured such that the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein, in a process of spraying the molten urea in an interior of the urea prilling tower and taking in an atmosphere at an ambient temperature of the urea prilling tower through the atmosphere inlet ports upwardly from below to cool and solidify the molten urea to form the urea particles,
heating air at a temperature higher than the ambient temperature is supplied in addition to the atmosphere at the ambient temperature to heat the interior of the urea prilling tower.

(2) A method for prilling urea using a urea prilling tower including an exhaust port provided at a tower top, a spraying portion for molten urea, a plurality of atmosphere inlet ports and a collection portion for prilled urea particles, in which
the urea prilling tower is configured such that the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein, in a process of spraying the molten urea in an interior of the urea prilling tower and taking in an atmosphere at an ambient temperature of the urea prilling tower through the atmosphere inlet ports upwardly from below to cool and solidify the molten urea to form the urea particles,
a temperature when an environmental temperature of surroundings in which the urea prilling tower is installed (the ambient temperature) is highest is considered to be an assumed highest design temperature of the urea prilling tower, and when the ambient temperature at which the urea prilling tower is installed is lower than the assumed highest temperature, a step of supplying heating air at a temperature higher than the ambient temperature in addition to the atmosphere at the ambient temperature to heat the interior of the urea prilling tower is performed.

(3) A method for prilling urea using a urea prilling tower including an exhaust port provided at a tower top, a spraying portion for molten urea, a plurality of atmosphere inlet ports and a collection portion for prilled urea particles, in which
the urea prilling tower is configured such that the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein, in a process of spraying the molten urea in an interior of the urea prilling tower and taking in an atmosphere at an ambient temperature of the urea prilling tower through the atmosphere inlet ports upwardly from below to cool and solidify the molten urea to form the urea particles,
when heating air at a temperature higher than the ambient temperature is supplied in addition to the atmosphere at the ambient temperature to heat the interior of the urea prilling tower, and when an operation load factor in the urea prilling tower (a percentage of an average urea particle production amount during a period to a maximum urea particle production amount during the same period) is a low value (Lₓ) (%) as compared to a maximum value (Lₘₐₓ) (%), a step of increasing a supply amount of the heating air as a difference of Lₘₐₓ−Lₓ (%) increases is performed.

(4) The method for prilling urea according to any one of (1) to (3), wherein when a supply amount of the heating air (temperature T° C.) to the interior of the urea prilling tower when Lₘₐₓ=100% is V, a supply amount of the heating air (temperature T° C.) when Lₓ=50% falls within the range of 1V to 5V.

(5) The method for prilling urea according to any one of (1) to (3), wherein the step of supplying the heating air at a temperature higher than the ambient temperature in addition to the atmosphere at the ambient temperature is any one step selected from:
(a) a step of supplying the heating air from the outside of the atmosphere inlet ports while taking in the atmosphere through the atmosphere inlet ports to the interior;
(b) a step of supplying part of the heating air from the outside of the atmosphere inlet ports and supplying the rest of the heating air directly to the inside of the atmosphere inlet ports while taking in the atmosphere through the atmosphere inlet ports to the interior; and
(c) a step of supplying the heating air directly to the inside of the atmosphere inlet ports while taking in the atmosphere through the atmosphere inlet ports to the interior.

(6) The method for prilling urea according to (5), wherein the step of supplying the heating air at a temperature higher than the ambient temperature in addition to the atmosphere at the ambient temperature is the step of (a) or (b), and is one step selected from:
(ab-1) a step of supplying the heating air from a plurality of positions opposed to the plurality of atmosphere inlet ports when supplying the heating air from the outside of the atmosphere inlet ports; and
(ab-2) a step of supplying the heating air from a plurality of positions outside the urea prilling tower and between the plurality of atmosphere inlet ports towards adjacent atmosphere inlet ports on both sides when supplying the heating air to the atmosphere inlet ports.

(7) The method for prilling urea according to any one of (1) to (3), wherein when the heating air at a temperature higher than the ambient temperature is supplied in addition to the atmosphere at the ambient temperature, the atmosphere at the ambient temperature is heated with a heater or a heat exchanger and supplied.

(8) The method for prilling urea according to any one of (1) to (3),
wherein a first requirement that a temperature of the urea particles immediately after collection from the collection portion of the urea prilling tower is 40° C. or more is satisfied,
a second requirement that a moisture content of the urea particles immediately after collection from the collection portion of the urea prilling tower (measurement method: measuring with a Karl Fischer moisture meter or heating an analysis sample with a drier and measuring by loss on drying) is 0.3% or less is satisfied, or
both the first and second requirements are satisfied.

(9) A urea prilling apparatus including a urea prilling tower and a heating air supply device, in which
the urea prilling tower includes an exhaust port provided at a tower top, a spraying portion for molten urea, a plurality of atmosphere inlet ports and a collection portion for prilling urea particles, and
the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein the heating air supply device includes a heating air production portion and a heating air supply line for supplying heating air produced in the heating air production portion to an interior of the urea prilling tower.

(10) A urea prilling apparatus including a urea prilling tower and a heating air supply device, in which
the urea prilling tower includes an exhaust port provided at a tower top, a spraying portion for molten urea, a plurality of atmosphere inlet ports and a collection portion for prilled urea particles, and
the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein each of the plurality of atmosphere inlet ports includes an adjustment tool for an atmospheric inflow rate and inflow direction, and the adjustment tool increases or reduces an opening area of the atmosphere inlet port to adjust the inflow rate through the atmosphere inlet port, the inflow direction through the atmosphere inlet port to an interior or both the inflow rate and the inflow direction, and
the heating air supply device includes a heating air production portion and a heating air supply line for supplying heating air produced in the heating air production portion through the atmosphere inlet ports to an interior of the urea prilling tower.

(11) A urea prilling apparatus including a urea prilling tower and a heating air supply device, in which
the urea prilling tower includes an exhaust port provided at a tower top, a spraying portion for molten urea, a plurality of atmosphere inlet ports and a collection portion for prilled urea particles, and
the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein each of the plurality of atmosphere inlet ports includes an adjustment tool for an atmospheric inflow rate and inflow direction,
the adjustment tool for the inflow rate and inflow direction is a louver made of a combination of multiple sets of a rotatable rotation shaft and a vane fixed to the rotation shaft,
the rotation shaft has ends of the rotation shaft attached respectively to two opposed positions of the atmosphere inlet port in a rotatable manner such that the rotation of the rotation shaft changes an angle formed between a plane of the atmosphere inlet port and a surface of the vane between 0 degrees and 90 degrees to adjust the inflow rate through the atmosphere inlet port, the inflow direction through the atmosphere inlet port to an interior or both the inflow rate and the inflow direction, and
the heating air supply device includes a heating air production portion and a heating air supply line for supplying heating air produced in the heating air production portion through the atmosphere inlet ports to an interior of the urea prilling tower.

(12) A urea prilling apparatus including a urea prilling tower and a heating air supply device, in which
the urea prilling tower includes an exhaust port provided at a tower top, a spraying portion for molten urea, a plurality of atmosphere inlet ports and a collection portion for prilled urea particles, and
the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein each of the plurality of atmosphere inlet ports includes an adjustment tool for an atmospheric inflow rate and inflow direction,
the adjustment tool for the atmospheric inflow rate and inflow direction is a shutter attached to an upper end, a lower end or another portion of the atmosphere inlet port and capable of reducing or increasing an opening area of the atmosphere inlet port,
the shutter reduces or increases the opening area of the atmosphere inlet port to adjust the inflow rate through the atmosphere inlet port, the inflow direction to an interior or both the inflow rate and the inflow direction, and
the heating air supply device includes a heating air production portion and a heating air supply line for supplying heating air produced in the heating air production portion through the atmosphere inlet ports to an interior of the urea prilling tower.

(13) The urea prilling apparatus according to any one of (9) to (12), wherein the urea prilling tower and the heating air supply device are arranged in such a manner that a heating air discharge port of the heating air production portion is connected to a first end opening of the heating air supply line, and a second end opening of the heating air supply line is spaced and disposed opposite to the plurality of atmosphere inlet ports of the urea prilling tower or directly connected thereto.

(14) The urea prilling apparatus according to any one of (9) to (12),
wherein the urea prilling tower and the heating air supply device are arranged in such a manner that a heating air discharge port of the heating air production portion is connected to a first end opening of the heating air supply line, and a second end opening of the heating air supply line is connected to the plurality of atmosphere inlet ports of the urea prilling tower,
the second end opening of the heating air supply line is branched to form a plurality of branch lines, and has any form selected from:
a first form in which respective openings of the plurality of branch lines are disposed opposite to the plurality of atmosphere inlet ports;
a second form in which the respective openings of the plurality of branch lines are directly connected to the plurality of atmosphere inlet ports; and
a third form which includes both the first and second forms.

(15) The urea prilling apparatus according to any one of (9) to (12),
wherein the urea prilling tower and the heating air supply device are arranged in such a manner that a heating air discharge port of the heating air production portion is connected to a first end opening of the heating air supply line, and a second end opening of the heating air supply line is connected to the plurality of atmosphere inlet ports of the urea prilling tower,
the second end opening of the heating air supply line includes an annular portion through which the heating air flows, and a plurality of hollow columnar portions disposed at intervals in a circumferential direction of the annular portion and provided vertically from the annular portion in a ventilatable manner, and the hollow columnar portions each include a slit-like opening in a portion thereof, or the hollow columnar portions each include a group of multiple pores in a portion thereof,
the plurality of atmosphere inlet ports and the plurality of hollow columnar portions including the slit-like opening or the group of multiple pores are arranged in such a manner that the plurality of atmosphere inlet ports are disposed on an inner side and the plurality of hollow columnar portions are disposed on an outer side, and have any form selected from:
a first form in which the slit-like openings or the groups of multiple pores of the plurality of hollow columnar portions are disposed opposite to the plurality of atmosphere inlet ports;
a second form in which the plurality of hollow columnar portions including the slit-like opening or the group of multiple pores are directly connected to the plurality of atmosphere inlet ports; and
a third form in which the plurality of atmosphere inlet ports and the plurality of hollow columnar portions including the slit-like opening or the group of multiple pores include both the first and second forms, and
further, when the first to third forms are viewed from the outside, the plurality of atmosphere inlet ports on the inner side and the plurality of hollow columnar portions on the outer side are disposed in an overlapping manner such that the slit-like openings or the groups of multiple pores of the hollow columnar portions are positioned within the range of the atmosphere inlet ports.

(16) The urea prilling apparatus according to any one of (9) to (12),
wherein the urea prilling tower and the heating air supply device are arranged in such a manner that a heating air discharge port of the heating air production portion is connected to a first end opening of the heating air supply line, and a second end opening of the heating air supply line is connected to the plurality of atmosphere inlet ports of the urea prilling tower,
the second end opening of the heating air supply line includes an annular portion through which the heating air flows, and a plurality of hollow columnar portions disposed at intervals in a circumferential direction of the annular portion and provided vertically from the annular portion, and the hollow columnar portions each include a plurality of slit-like openings or groups of multiple pores formed at two different positions thereof,
the plurality of atmosphere inlet ports and the plurality of hollow columnar portions including the slit-like openings or the groups of multiple pores have a first form in which they are disposed to allow the slit-like openings or the groups of multiple pores formed at the two different positions of the plurality of hollow columnar portions to release the heating air between adjacent atmosphere inlet ports of the plurality of atmosphere inlet ports towards the adjacent atmosphere inlet ports, and
in addition to the first form, further have as necessary, a second form in which second hollow columnar portions including an opening which are branched from the plurality of hollow columnar portions including the slit-like openings or the groups of multiple pores and extended in a direction along the annular portion are directly connected to the atmosphere inlet ports, or
a third form in which a plurality of third hollow columnar portions including an opening are provided vertically from the annular portion, and the openings of the third hollow columnar portions are disposed opposite to the atmosphere inlet ports and directly connected thereto,
in the second and third forms, the heating air is released from a lower side of the plurality of atmosphere inlet ports, and
further, adjustment tools for an inflow rate and an inflow direction included in the adjacent atmosphere inlet ports are adjusted to allow the heating air released from the slit-like openings or the groups of multiple pores formed at the two different positions of the plurality of hollow columnar portions to flow into the interior.

(17) The urea prilling apparatus according to any one of (9) to (12), wherein the heating air production portion of the heating air supply device includes a heater or a heat exchanger for heating an atmosphere taken in, and a blowing device for blowing the heated atmosphere.

(18) The urea prilling apparatus according to any one of (9) to (12), wherein the urea prilling apparatus includes any one selected from:
(A) a combination of a single urea prilling tower and a single heating air supply device;
(B) a combination of a single urea prilling tower and a plurality of heating air supply devices;
(C) a combination of a plurality of urea prilling towers and a single heating air supply device; and
(D) a combination of a plurality of urea prilling towers and a plurality of heating air supply devices.

(19) The urea prilling apparatus according to any one of (9) to (12), wherein a temperature when an environmental temperature of surroundings in which the urea prilling tower is installed (an ambient temperature) is highest is considered to be an assumed highest design temperature of the urea prilling tower, and when the ambient temperature at which the urea prilling tower is installed is lower than the assumed highest temperature, the heating air at a temperature higher than the ambient temperature is supplied in addition to an atmosphere at the ambient temperature to heat the interior of the urea prilling tower.

(20) The urea prilling apparatus according to any one of (9) to (12), wherein a temperature when an environmental temperature of surroundings in which the urea prilling tower is installed (an ambient temperature) is highest is considered to be an assumed highest design temperature of the urea prilling tower, and when the ambient temperature at which the urea prilling tower is installed is lower than the assumed highest temperature, the heating air at a temperature higher than the ambient temperature is supplied in addition to an atmosphere at the ambient temperature to heat the interior of the urea prilling tower, and
when an operation load factor in the urea prilling tower (a percentage of an average urea particle production amount during a period to a maximum urea particle production amount during the same period) is a low value (Lₓ) (%) as compared to a maximum value (Lₘₐₓ) (%), a supply amount of the heating air is increased as a difference of Lₘₐₓ−Lₓ (%) increases.

(21) The urea prilling apparatus according to any one of (9) to (12),
wherein a first requirement that a temperature of the urea particles immediately after collection from the collection portion of the urea prilling tower can be 40° C. or more can be satisfied,
a second requirement that a moisture content of the urea particles immediately after collection from the collection portion of the urea prilling tower (measurement method: measuring with a Karl Fischer moisture meter or heating an analysis sample with a drier and measuring by loss on drying) can be 0.3% or less can be satisfied, or
both the first and second requirements can be satisfied.

(22) The urea prilling apparatus according to any one of (9) to (12), wherein the apparatus is not provided with an acid scrubbing facility for processing exhaust air from the exhaust port provided at the tower top of the urea prilling tower.

(23) A method for prilling urea using a urea prilling apparatus including a urea prilling tower and a heating air supply device, in which
the urea prilling tower includes an exhaust port provided at a tower top, a spraying portion for molten urea, a plurality of atmosphere inlet ports and a collection portion for prilled urea particles, and
the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein the heating air supply device includes a heating air production portion and a heating air supply line for supplying heating air produced in the heating air production portion to an interior of the urea prilling tower, and
wherein the method for prilling urea includes
a first step of spraying the molten urea from the spraying portion of the urea prilling tower and taking in an atmosphere at an ambient temperature of the urea prilling tower through the plurality of atmosphere inlet ports upwardly from below to cool and solidify the molten urea to form the urea particles, and
a second step of recovering the urea particles produced in the previous step from the collection portion, and
the first step includes a step of supplying, in addition to the atmosphere at the ambient temperature, the heating air at a temperature higher than the ambient temperature from the heating air supply device to the plurality of atmosphere inlet ports to heat the interior of the urea prilling tower.

(24) A method for prilling urea using a urea prilling apparatus including a urea prilling tower and a heating air supply device, in which
the urea prilling tower includes an exhaust port provided at a tower top, a spraying portion for molten urea, a plurality of atmosphere inlet ports and a collection portion for prilled urea particles, and
the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein each of the plurality of atmosphere inlet ports includes an adjustment tool for an atmospheric inflow rate and inflow direction, and the adjustment tool increases or reduces an opening area of the atmosphere inlet port to adjust the inflow rate through the atmosphere inlet port, the inflow direction through the atmosphere inlet port to an interior or both the inflow rate and the inflow direction, and
the heating air supply device includes a heating air production portion and a heating air supply line for supplying heating air produced in the heating air production portion through the atmosphere inlet ports to an interior of the urea prilling tower, and
wherein the method for prilling urea includes
a first step of spraying the molten urea from the spraying portion of the urea prilling tower and taking in an atmosphere at an ambient temperature of the urea prilling tower through the plurality of atmosphere inlet ports upwardly from below to cool and solidify the molten urea to form the urea particles, and
a second step of recovering the urea particles produced in the previous step from the collection portion, and
when supplying, in addition to the atmosphere at the ambient temperature, the heating air at a temperature higher than the ambient temperature from the heating air supply device to the plurality of atmosphere inlet ports to heat the interior of the urea prilling tower, the first step includes a step of increasing or reducing the opening area of the atmosphere inlet port by the adjustment tool for the atmospheric inflow rate and inflow direction to adjust the inflow rate through the atmosphere inlet port, the inflow direction through the atmosphere inlet port to the interior or both the inflow rate and the inflow direction.

(25) A method for prilling urea using a urea prilling apparatus including a urea prilling tower and a heating air supply device, in which
the urea prilling tower includes an exhaust port provided at a tower top, a spraying portion for molten urea, a plurality of atmosphere inlet ports and a collection portion for prilled urea particles, and
the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein each of the plurality of atmosphere inlet ports includes an adjustment tool for an atmospheric inflow rate and inflow direction,
the adjustment tool for the atmospheric inflow rate and inflow direction is a louver made of a combination of multiple sets of a rotatable rotation shaft and a vane fixed to the rotation shaft,
the rotation shaft has ends of the rotation shaft attached respectively to two opposed positions of the atmosphere inlet port in a rotatable manner such that the rotation of the rotation shaft changes an angle formed between a plane of the atmosphere inlet port and a surface of the vane between 0 degrees and 90 degrees to adjust the inflow rate through the atmosphere inlet port, the inflow direction through the atmosphere inlet port to an interior or both the inflow rate and the inflow direction, and
the heating air supply device includes a heating air production portion and a heating air supply line for supplying heating air produced in the heating air production portion through the atmosphere inlet ports to an interior of the urea prilling tower, and
wherein the method for prilling urea includes
a first step of spraying the molten urea from the spraying portion of the urea prilling tower and taking in an atmosphere at an ambient temperature of the urea prilling tower through the plurality of atmosphere inlet ports upwardly from below to cool and solidify the molten urea to form the urea particles, and
a second step of recovering the urea particles produced in the previous step from the collection portion, and
when supplying, in addition to the atmosphere at the ambient temperature, the heating air at a temperature higher than the ambient temperature from the heating air supply device to the plurality of atmosphere inlet ports to heat the interior of the urea prilling tower, the first step includes a step of increasing or reducing the opening area of the atmosphere inlet port by the adjustment tool for the atmospheric inflow rate and inflow direction to adjust the inflow rate through the atmosphere inlet port, the inflow direction through the atmosphere inlet port to the interior or both the inflow rate and the inflow direction,
the adjustment tool for the atmospheric inflow rate and inflow direction is a louver made of a combination of multiple sets of a rotatable rotation shaft and a vane fixed to the rotation shaft, and the rotation shaft has ends of the rotation shaft attached respectively to two opposed positions of the atmosphere inlet port in a rotatable manner, and
the rotation of the rotation shaft changes an angle formed between a plane of the atmosphere inlet port and a surface of the vane between 0 degrees and 90 degrees to increase or reduce the opening area of the atmosphere inlet port to adjust the inflow rate through the atmosphere inlet port, the inflow direction through the atmosphere inlet port to the interior or both the inflow rate and the inflow direction.

(26) A method for prilling urea using a urea prilling apparatus including a urea prilling tower and a heating air supply device, in which
the urea prilling tower includes an exhaust port provided at a tower top, a spraying portion for molten urea, a plurality of atmosphere inlet ports and a collection portion for prilled urea particles, and
the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein each of the plurality of atmosphere inlet ports includes an adjustment tool for an atmospheric inflow rate and inflow direction,
the adjustment tool for the atmospheric inflow rate and inflow direction is a shutter attached to an upper end, a lower end or another portion of the atmosphere inlet port and capable of reducing or increasing an opening area of the atmosphere inlet port,
the shutter reduces or increases the opening area of the atmosphere inlet port to adjust the inflow rate through the atmosphere inlet port, the inflow direction to an interior or both the inflow rate and the inflow direction, and
the heating air supply device includes a heating air production portion and a heating air supply line for supplying heating air produced in the heating air production portion through the atmosphere inlet ports to an interior of the urea prilling tower, and
wherein the method for prilling urea includes
a first step of spraying the molten urea from the spraying portion of the urea prilling tower and taking in an atmosphere at an ambient temperature of the urea prilling tower through the plurality of atmosphere inlet ports upwardly from below to cool and solidify the molten urea to form the urea particles, and
a second step of recovering the urea particles produced in the previous step from the collection portion, and
when supplying, in addition to the atmosphere at the ambient temperature, the heating air at a temperature higher than the ambient temperature from the heating air supply device to the plurality of atmosphere inlet ports to heat the interior of the urea prilling tower, the first step includes a step of increasing or reducing the opening area of the atmosphere inlet port by the adjustment tool for the atmospheric inflow rate and inflow direction to adjust the inflow rate through the atmosphere inlet port, the inflow direction through the atmosphere inlet port to the interior or both the inflow rate and the inflow direction,
the adjustment tool for the atmospheric inflow rate and inflow direction is a shutter attached to an upper end, a lower end or another portion of the atmosphere inlet port and capable of reducing or increasing the opening area of the atmosphere inlet port, and
the shutter reduces or increases the opening area of the atmosphere inlet port to adjust the inflow rate through the atmosphere inlet port, the inflow direction to the interior or both the inflow rate and the inflow direction.

(27) The method for prilling urea using a urea prilling apparatus according to any one of (23) to (26), wherein the urea prilling tower and the heating air supply device are arranged in such a manner that a heating air discharge port of the heating air production portion is connected to a first end opening of the heating air supply line, and a second end opening of the heating air supply line is spaced and disposed opposite to the plurality of atmosphere inlet ports of the urea prilling tower or directly connected thereto.

(28) The method for prilling urea using a urea prilling apparatus according to any one of (23) to (26),
wherein the urea prilling tower and the heating air supply device are arranged in such a manner that a heating air discharge port of the heating air production portion is connected to a first end opening of the heating air supply line, and a second end opening of the heating air supply line is connected to the plurality of atmosphere inlet ports of the urea prilling tower, and
the second end opening of the heating air supply line is branched to form a plurality of branch lines, and has any form selected from:
a first form in which respective openings of the plurality of branch lines are disposed opposite to the plurality of atmosphere inlet ports;
a second form in which the respective openings of the plurality of branch lines are directly connected to the plurality of atmosphere inlet ports; and a third form which includes both the first and second forms.

(29) The method for prilling urea using a urea prilling apparatus according to any one of (23) to (26),
wherein the urea prilling tower and the heating air supply device are arranged in such a manner that a heating air discharge port of the heating air production portion is connected to a first end opening of the heating air supply line, and a second end opening of the heating air supply line is connected to the plurality of atmosphere inlet ports of the urea prilling tower,
the second end opening of the heating air supply line includes an annular portion through which the heating air flows, and a plurality of hollow columnar portions disposed at intervals in a circumferential direction of the annular portion and provided vertically from the annular portion in a ventilatable manner, and the hollow columnar portions each include a slit-like opening in a portion thereof, or the hollow columnar portions each include a group of multiple pores in a portion thereof,
the plurality of atmosphere inlet ports and the plurality of hollow columnar portions including the slit-like opening or the group of multiple pores are arranged in such a manner that the plurality of atmosphere inlet ports are disposed on an inner side and the plurality of hollow columnar portions are disposed on an outer side, and have any form selected from:
a first form in which the slit-like openings or the groups of multiple pores of the plurality of hollow columnar portions are disposed opposite to the plurality of atmosphere inlet ports;
a second form in which the plurality of hollow columnar portions including the slit-like opening or the group of multiple pores are directly connected to the plurality of atmosphere inlet ports; and
a third form in which the plurality of atmosphere inlet ports and the plurality of hollow columnar portions including the slit-like opening or the group of multiple pores include both the first and second forms, and
further, when the first to third forms are viewed from the outside, the plurality of atmosphere inlet ports on the inner side and the plurality of hollow columnar portions on the outer side are disposed in an overlapping manner such that the slit-like openings or the groups of multiple pores of the hollow columnar portions are positioned within the range of the atmosphere inlet ports.

(30) The method for prilling urea using a urea prilling apparatus according to any one of (23) to (26),
wherein the urea prilling tower and the heating air supply device are arranged in such a manner that a heating air discharge port of the heating air production portion is connected to a first end opening of the heating air supply line, and a second end opening of the heating air supply line is connected to the plurality of atmosphere inlet ports of the urea prilling tower,
the second end opening of the heating air supply line includes an annular portion through which the heating air flows, and a plurality of hollow columnar portions disposed at intervals in a circumferential direction of the annular portion and provided vertically from the annular portion, and the hollow columnar portions each include a plurality of slit-like openings or groups of multiple pores formed lengthwise at two different positions thereof,
the plurality of atmosphere inlet ports and the plurality of hollow columnar portions including the slit-like openings or the groups of multiple pores are disposed to allow the slit-like openings or the groups of multiple pores formed at the two different positions of the plurality of hollow columnar portions to release the heating air between adjacent atmosphere inlet ports of the plurality of atmosphere inlet ports towards the adjacent atmosphere inlet ports, and
further, adjustment tools for an inflow rate and an inflow direction included in the adjacent atmosphere inlet ports are adjusted to allow the heating air released from the slit-like openings or the groups of multiple pores formed at the two different positions of the plurality of hollow columnar portions to flow into the interior.

(31) The method for prilling urea using a urea prilling apparatus according to any one of (23) to (26), wherein the heating air production portion of the heating air supply device includes a heater or a heat exchanger for heating an atmosphere taken in, and a blowing device for blowing the heated atmosphere.

(32) The method for prilling urea using a urea prilling apparatus according to any one of (23) to (26), wherein the urea prilling apparatus includes any one selected from:
(A) a combination of a single urea prilling tower and a single heating air supply device;
(B) a combination of a single urea prilling tower and a plurality of heating air supply devices;
(C) a combination of a plurality of urea prilling towers and a single heating air supply device; and
(D) a combination of a plurality of urea prilling towers and a plurality of heating air supply devices.

(33) The method for prilling urea using a urea prilling apparatus according to any one of (23) to (26), wherein a temperature when an environmental temperature of surroundings in which the urea prilling tower is installed (the ambient temperature) is highest is considered to be an assumed highest design temperature of the urea prilling tower, and when the ambient temperature at which the urea prilling tower is installed is lower than the assumed highest temperature, the heating air at a temperature higher than the ambient temperature is supplied in addition to the atmosphere at the ambient temperature to heat the interior of the urea prilling tower.

(34) The method for prilling urea using a urea prilling apparatus according to any one of (23) to (26), wherein the heating air at a temperature higher than the ambient temperature is supplied in addition to the atmosphere at the ambient temperature to heat the interior of the urea prilling tower, and
when an operation load factor in the urea prilling tower (a percentage of an average urea particle production amount during a period to a maximum urea particle production amount during the same period) is a low value (Lₓ) (%) as compared to a maximum value (Lₘₐₓ) (%), a supply amount of the heating air is increased as a difference of Lₘₐₓ−Lₓ (%) increases.

(35) The method for prilling urea using a urea prilling apparatus according to any one of (23) to (26), wherein the method for prilling urea using a urea prilling apparatus can satisfy
a first requirement that a temperature of the urea particles immediately after the collection from the collection portion of the urea prilling tower can be 40° C. or more,
a second requirement that a moisture content of the urea particles immediately after the collection from the collection portion of the urea prilling tower (measurement method: measuring with a Karl Fischer moisture meter or heating an analysis sample with a drier and measuring by loss on drying) can be 0.3% or less, or
both the first and second requirements.

(36) The method for prilling urea using a urea prilling apparatus according to any one of (23) to (26), wherein the method for prilling urea using a urea prilling apparatus is a method not performing acid scrubbing for processing exhaust air.

### EXAMPLES

### Comparative Example 1

Urea was prilled using the urea prilling apparatus shown in FIGS. 1 to 3 (excluding the heating air supply device 30). The operation loads in Table 1 are operation load factors in the urea prilling tower, and the same applies to Tables 2 to 5. The results are shown in Table 1.

TABLE 1

| | | | | |
|---|---|---|---|---|
| Production | Ton/Day | 2,750 | 2,063 | 1,375 |
| amount | | | | |
| Operation | % | 100% | 75% | 50% |
| load | | | | |
| Atmospheric | deg. C. | 35 | 35 | 35 |
| temperature | | | | |
| Amount of | Nm³/h | 1,531,000 | 1,531,000 | 1,531,000 |
| atmosphere | | | | |
| taken in | | | | |
| Product | deg. C. | 50 | 48 | 47 |
| temperature | | | | |

### Example 1

Urea was prilled using the urea prilling apparatus shown in FIGS. 1 to 3. The prilling conditions were the same as in the prilling method of comparative example 1 except that the heating air was introduced through the atmosphere inlet ports. Note that the atmospheric temperature 35 deg.C was considered to be “an assumed highest design temperature of the urea prilling tower, a temperature when an environmental temperature of surroundings in which the urea prilling tower is installed (ambient temperature) is highest.” The results are shown in Table 2.

TABLE 2

| | | | | |
|---|---|---|---|---|
| Production amount | Ton/Day | 2,750 | 2,063 | 1,375 |
| Operation load | % | 100% | 75% | 50% |
| Atmospheric temperature | deg. C. | 35 | 35 | 35 |
| Amount of atmosphere taken in | Nm³/h | 1,531,000 | 1,443,000 | 1,399,000 |
| Amount of heating air | Nm³/h | 0 | 88,000 | 132,000 |
| Amount of atmosphere taken | Nm³/h | 1,531,000 | 1,531,000 | 1,531,000 |
| in + amount of heating air | | | | |
| Heating air temperature | deg. C. | 70 | 70 | 70 |
| Temperature after mixing of | deg. C. | 35 | 37 | 38 |
| heating air/atmosphere | | | | |
| Product temperature | deg. C. | 50 | 50 | 50 |

The product of comparative example 1 in which the heating air was not supplied during prilling in the urea prilling tower was considered to have variations in quality (moisture content) in view of the fact that the product temperature decreased as the operation load decreased, as is clear from product temperature comparisons between Tables 1 and 2. On the other hand, the product of example 1 was considered to have stable quality (moisture content) in view of the fact that the product temperature was constant even if the operation load decreased.

### Comparative Example 2

Urea was prilled using the urea prilling apparatus shown in FIGS. 1 to 3 (excluding the heating air supply device 30). The results are shown in Table 3.

TABLE 3

| | | | | |
|---|---|---|---|---|
| Production | Ton/Day | 2,750 | 2,750 | 2,750 |
| amount | | | | |
| Operation | % | 100% | 100% | 100% |
| load | | | | |
| Atmospheric | deg. C. | 35 | 30 | 25 |
| temperature | | | | |
| Amount of | Nm³/h | 1,531,000 | 1,531,000 | 1,531,000 |
| atmosphere | | | | |
| taken in | | | | |
| Product | deg. C. | 50 | 44 | 38 |
| temperature | | | | |

### Example 2

Urea was prilled using the urea prilling apparatus shown in FIGS. 1 to 3. The prilling conditions were the same as in the prilling method of comparative example 2 except that the heating air was introduced through the atmosphere inlet ports. Note that the atmospheric temperature 35 deg.C was considered to be “an assumed highest design temperature of the urea prilling tower, a temperature when an environmental temperature of surroundings in which the urea prilling tower is installed (ambient temperature) is highest.” The results are shown in Table 4.

TABLE 4

| | | | | |
|---|---|---|---|---|
| Production amount | Ton/Day | 2,750 | 2,750 | 2,750 |
| Operation load | % | 100% | 100% | 100% |
| Atmospheric temperature | deg. C. | 35 | 30 | 25 |
| Amount of atmosphere taken in | Nm³/h | 1,531,000 | 1,339,000 | 1,190,000 |
| Amount of heating air | Nm³/h | 0 | 192,000 | 341,000 |
| Amount of atmosphere taken | Nm³/h | 1,531,000 | 1,531,000 | 1,531,000 |
| in + amount of heating air | | | | |
| Heating air temperature | deg. C. | 70 | 70 | 70 |
| Temperature after mixing of | deg. C. | 35 | 35 | 35 |
| heating air/atmosphere | | | | |
| Product temperature | deg. C. | 50 | 50 | 50 |

The product of comparative example 2 in which the heating air was not supplied during prilling in the urea prilling tower was considered to have variations in quality (moisture content) in view of the fact that the product temperature decreased as the atmospheric temperature (temperature of a surrounding environment of the urea prilling tower) decreased, as is clear from product temperature comparisons between Tables 3 and 4. On the other hand, the product of example 2 was considered to have stable quality (moisture content) in view of the fact that the product temperature was constant even if the atmospheric temperature decreased.

### Comparative Example 3

Urea was prilled using the urea prilling apparatus shown in FIGS. 1 to 3 (excluding the heating air supply device 30). The results are shown in Table 5.

TABLE 5

| | | | | |
|---|---|---|---|---|
| Production | Ton/Day | 2,750 | 2,750 | 2,750 |
| amount | | | | |
| Operation load | % | 100% | 100% | 100% |
| Atmospheric | deg. C. | 35 | 30 | 25 |
| temperature | | | | |
| Amount of | Nm³/h | 1,531,000 | 1,058,000 | 793,000 |
| atmosphere | | | | |
| taken in | | | | |
| Product | deg. C. | 50 | 50 | 50 |
| temperature | | | | |
| Exhaust NH₃ | mg/Nm³ | 50 | 72 | 97 |
| concentration | | | | |

As is clear from Table 5, when urea is prilled with the production amount and operation load kept constant, even if the atmospheric temperature decreases, operation without reducing the product temperature is possible if the amount of the atmosphere taken in is reduced, but there is a problem of the increased ammonia concentrations in exhaust air. In such a case, an acid scrubbing facility for processing the exhaust air is required as an environmental measure, but it is undesirable because other problems such as increased production costs and generation of by-products arise.

### INDUSTRIAL APPLICABILITY

The method for prilling urea of the present invention can be utilized as a method for producing urea particles of stable quality.

### REFERENCE SIGNS LIST

1 Urea prilling apparatus
10 Urea prilling tower
11 Tower top
12 Tower bottom
13 Tower side wall
14 Exhaust port
15 Spraying portion for molten urea
15a Supply line for molten urea
16 (16a~16h) Atmosphere inlet port
17 Collection portion for urea particles
30 Heating air supply device
31 Heating air production portion
40 Heating air supply line
42 Annular portion
43 (43a~43h) Heating air discharge portion

1. A method for prilling urea using a urea prilling tower including an exhaust port provided at a tower top, a spraying portion for molten urea, a plurality of atmosphere inlet ports and a collection portion for prilled urea particles, in which
the urea prilling tower is configured such that the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein, in a process of spraying the molten urea in an interior of the urea prilling tower, taking in an atmosphere at an ambient temperature of the urea prilling tower through the atmosphere inlet ports upwardly from below to cool and solidify the molten urea to form the urea particles,
heating an interior of the urea prilling tower by supplying heating air having a temperature higher than the ambient temperature,
the heating air at a temperature higher than the ambient temperature being supplied in addition to the atmosphere at the ambient temperature to heat the interior of the urea prilling tower.

2. The method for prilling urea according to claim 1,
wherein the heating air at a temperature higher than the ambient temperature is supplied in addition to the atmosphere at the ambient temperature to heat the interior of the urea prilling tower when the ambient temperature at which the urea prilling tower is installed is lower than an assumed highest temperature of the urea prilling tower, the assumed highest temperature of the urea prilling tower being a design temperature at which an environmental temperature of surroundings in which the urea prilling tower is installed is highest.

3. The method for prilling urea according to claim 1,
wherein
when the heating air at a temperature higher than the ambient temperature is supplied in addition to the atmosphere at the ambient temperature to heat the interior of the urea prilling tower, and provided that an operation load factor in the urea prilling tower being a percentage of an average urea particle production amount during a period to a maximum urea particle production amount during the same period is at a low value (Lₓ) (%) as compared to a maximum value (Lₘₐₓ) (%), a step of increasing a supply amount of the heating air as a difference of Lₘₐₓ−Lₓ (% increases is performed.

4. The method for prilling urea according to claim 1, wherein the step of supplying the heating air at a temperature higher than the ambient temperature in addition to the atmosphere at the ambient temperature is any one step selected from:
(a) a step of supplying the heating air from the outside of the atmosphere inlet ports while taking in the atmosphere through the atmosphere inlet ports to the interior;
(b) a step of supplying part of the heating air from the outside of the atmosphere inlet ports and supplying the rest of the heating air directly to the inside of the atmosphere inlet ports while taking in the atmosphere through the atmosphere inlet ports to the interior; and
(c) a step of supplying the heating air directly to the inside of the atmosphere inlet ports while taking in the atmosphere through the atmosphere inlet ports to the interior.

5. The method for prilling urea according to claim 4, wherein the step of supplying the heating air at a temperature higher than the ambient temperature in addition to the atmosphere at the ambient temperature is the step of (a) or (b), and is one step selected from:
(ab-1) a step of supplying the heating air from a plurality of positions opposed to the plurality of atmosphere inlet ports when supplying the heating air from the outside of the atmosphere inlet ports; and
(ab-2) a step of supplying the heating air from a plurality of positions outside the urea prilling tower and between the plurality of atmosphere inlet ports towards adjacent atmosphere inlet ports on both sides when supplying the heating air to the atmosphere inlet ports.

6. The method for prilling urea according to claim 1, wherein when the heating air at a temperature higher than the ambient temperature is supplied in addition to the atmosphere at the ambient temperature, the atmosphere at the ambient temperature is heated with a heater or a heat exchanger and supplied.

7. The method for prilling urea according to claim 1,
wherein a first requirement that a temperature of the urea particles immediately after collection from the collection portion of the urea prilling tower is 40° C. or more is satisfied,
a second requirement that a moisture content of the urea particles immediately after collection from the collection portion of the urea prilling tower (measurement method: measuring with a Karl Fischer moisture meter or heating an analysis sample with a drier and measuring by loss on drying) is 0.3% or less is satisfied, or
both the first and second requirements are satisfied.

8. A urea prilling apparatus comprising a urea prilling tower and a heating air supply device, in which
the urea prilling tower includes an exhaust port provided at a tower top, a spraying portion for molten urea, a plurality of atmosphere inlet ports and a collection portion for prilled urea particles, and
the spraying portion for molten urea is disposed on an upper side of the urea prilling tower, the plurality of atmosphere inlet ports are disposed on a lower side of the urea prilling tower, the collection portion is disposed at a tower bottom on a further lower side of the atmosphere inlet ports, and the plurality of atmosphere inlet ports are disposed at intervals in a circumferential direction,
wherein the heating air supply device includes a heating air production portion and a heating air supply line for supplying heating air produced in the heating air production portion to an interior of the urea prilling tower through the atmosphere inlet ports.

9. The urea prilling apparatus according to claim 8,
wherein each of the plurality of atmosphere inlet ports includes an adjustment tool for an atmospheric inflow rate and inflow direction, and the adjustment tool increases or reduces an opening area of the atmosphere inlet port to adjust the inflow rate through the atmosphere inlet port, the inflow direction through the atmosphere inlet port to the interior or both the inflow rate and the inflow direction.

10. The urea prilling apparatus according to claim 8,
wherein each of the plurality of atmosphere inlet ports includes an adjustment tool for an atmospheric inflow rate and inflow direction,
the adjustment tool for the inflow rate and inflow direction is a louver made of a combination of multiple sets of a rotatable rotation shaft and a vane fixed to the rotation shaft, and
the rotation shaft has ends of the rotation shaft attached respectively to two opposed positions of the atmosphere inlet port in a rotatable manner such that rotation of the rotation shaft changes an angle formed between a plane of the atmosphere inlet port and a surface of the vane between 0 degrees and 90 degrees to adjust the inflow rate through the atmosphere inlet port, the inflow direction through the atmosphere inlet port to the interior or both the inflow rate and the inflow direction.

11. The urea prilling apparatus according to claim 8,
wherein each of the plurality of atmosphere inlet ports includes an adjustment tool for an atmospheric inflow rate and inflow direction,
the adjustment tool for the inflow rate and inflow direction is a shutter attached to an upper end, a lower end or another portion of the atmosphere inlet port and is capable of reducing or increasing an opening area of the atmosphere inlet port, and
the shutter reduces or increases the opening area of the atmosphere inlet port to adjust the inflow rate through the atmosphere inlet port, the inflow direction to the interior or both the inflow rate and the inflow direction.

12. The urea prilling apparatus according to claim 8, wherein the urea prilling tower and the heating air supply device are arranged in such a manner that a heating air discharge port of the heating air production portion is connected to a first end opening of the heating air supply line, and a second end opening of the heating air supply line is spaced and disposed opposite to the plurality of atmosphere inlet ports of the urea prilling tower or directly connected thereto.

13. The urea prilling apparatus according to claim 8,
wherein the urea prilling tower and the heating air supply device are arranged in such a manner that a heating air discharge port of the heating air production portion is connected to a first end opening of the heating air supply line, and a second end opening of the heating air supply line is connected to the plurality of atmosphere inlet ports of the urea prilling tower, and
the second end opening of the heating air supply line is branched to form a plurality of branch lines, and has any form selected from:
a first form in which respective openings of the plurality of branch lines are disposed opposite to the plurality of atmosphere inlet ports;
a second form in which the respective openings of the plurality of branch lines are directly connected to the plurality of atmosphere inlet ports; and
a third form which includes both the first and second forms.

14. The urea prilling apparatus according to claim 8,
wherein the urea prilling tower and the heating air supply device are arranged in such a manner that a heating air discharge port of the heating air production portion is connected to a first end opening of the heating air supply line, and a second end opening of the heating air supply line is connected to the plurality of atmosphere inlet ports of the urea prilling tower,
the second end opening of the heating air supply line includes an annular portion through which the heating air flows, and a plurality of hollow columnar portions disposed at intervals in a circumferential direction of the annular portion and provided vertically from the annular portion in a ventilatable manner, and the hollow columnar portions each include a slit-like opening in a portion thereof, or the hollow columnar portions each include a group of multiple pores in a portion thereof,
the plurality of atmosphere inlet ports and the plurality of hollow columnar portions including the slit-like opening or the group of multiple pores are arranged in such a manner that the plurality of atmosphere inlet ports are disposed on an inner side and the plurality of hollow columnar portions are disposed on an outer side, and have any form selected from:
a first form in which the slit-like openings or the groups of multiple pores of the plurality of hollow columnar portions are disposed opposite to the plurality of atmosphere inlet ports;
a second form in which the plurality of hollow columnar portions including the slit-like opening or the group of multiple pores are directly connected to the plurality of atmosphere inlet ports; and
a third form in which the plurality of atmosphere inlet ports and the plurality of hollow columnar portions including the slit-like opening or the group of multiple pores include both the first and second forms, and
further, when the first to third forms are viewed from the outside, the plurality of atmosphere inlet ports on the inner side and the plurality of hollow columnar portions on the outer side are disposed in an overlapping manner such that the slit-like openings or the groups of multiple pores of the hollow columnar portions are positioned within the range of the atmosphere inlet ports.

15. The urea prilling apparatus according to claim 8,
wherein the urea prilling tower and the heating air supply device are arranged in such a manner that a heating air discharge port of the heating air production portion is connected to a first end opening of the heating air supply line, and a second end opening of the heating air supply line is connected to the plurality of atmosphere inlet ports of the urea prilling tower,
the second end opening of the heating air supply line includes an annular portion through which the heating air flows, and a plurality of hollow columnar portions disposed at intervals in a circumferential direction of the annular portion and provided vertically from the annular portion, and the hollow columnar portions each include a plurality of slit-like openings or groups of multiple pores formed lengthwise at two different positions thereof,
the plurality of atmosphere inlet ports and the plurality of hollow columnar portions including the slit-like openings or the groups of multiple pores have a first form in which they are disposed to allow the slit-like openings or the groups of multiple pores formed at the two different positions of the plurality of hollow columnar portions to release the heating air between adjacent atmosphere inlet ports of the plurality of atmosphere inlet ports towards the adjacent atmosphere inlet ports, and
in addition to the first form, further have as necessary, a second form in which second hollow columnar portions including an opening which are branched from the plurality of hollow columnar portions including the slit-like openings or the groups of multiple pores and extended in a direction along the annular portion are directly connected to the atmosphere inlet ports, or
a third form in which a plurality of third hollow columnar portions including an opening are provided vertically from the annular portion, and the openings of the third hollow columnar portions are directly connected to the atmosphere inlet ports,
in the second and third forms, the heating air is released from a lower side of the plurality of atmosphere inlet ports, and
further, adjustment tools for an inflow rate and an inflow direction included in the adjacent atmosphere inlet ports are adjusted to allow the heating air released from the slit-like openings or the groups of multiple pores formed at the two different positions of the plurality of hollow columnar portions to flow into the interior.

16. The urea prilling apparatus according to claim 8, wherein the heating air production portion of the heating air supply device comprises a heater or a heat exchanger for heating an atmosphere taken in, and a blowing device for blowing the heated atmosphere.

17. The urea prilling apparatus according to claim 8, wherein the urea prilling apparatus comprises any one selected from:
(A) a combination of a single urea prilling tower and a single heating air supply device;
(B) a combination of a single urea prilling tower and a plurality of heating air supply devices;
(C) a combination of a plurality of urea prilling towers and a single heating air supply device; and
(D) a combination of a plurality of urea prilling towers and a plurality of heating air supply devices.

18.-21. (canceled)
